(19) United States
(12) Patent Application Publication
(10) Pub. No.: US 2021/0001009 A1
(43) Pub. Date: Jan. 7, 2021

(54) BIOGUM AND BOTANICAL GUM HYDROGEL BIOINKS FOR THE PHYSIOLOGICAL 3D BIOPRINTING OF TISSUE CONSTRUCTS FOR IN VITRO CULTURE AND TRANSPLANTATION

(71) Applicant: Cellink AB, Gothenburg (SE)

(72) Inventors: Adel Itedale Namro Redwan, Skepplanda (SE); Patrick Thayer, Hudson, MA (US); Hector Martinez, Göteborg (SE); Erik Gatenholm, Gothenburg (SE)

(21) Appl. No.: 16/979,452

(22) PCT Filed: Oct. 25, 2019
(86) PCT No.: PCT/US19/58025

§ 371 Date: Sep. 9, 2020

Related U.S. Application Data
(60) Provisional application No. 62/750,390, filed on Oct. 25, 2018.
(60) Provisional application No. 62/750,417, filed on Oct. 25, 2018.

(51) Int. Cl. A61L 27/52; A61K 31/717; A61K 31/722; A61K 9/00; A61L 27/38; A61L 27/44

(52) U.S. Cl. CPC A61L 27/52; A61K 31/717; A61K 31/722; A61L 2400/06; A61L 27/38; A61L 27/44; A61K 9/0024

(57) ABSTRACT

Bioink compositions comprising a biomaterial (mammalian, plant based, synthetically derived, or microbially derived) such as a hydrogel and a microbial-, fungal-, or plant-produced polysaccharide, with or without cells, for use in the 3D bioprinting of human tissues and scaffolds are described. The bioink compositions have excellent printability and improved cell function, viability and engraftment. Furthermore, the bioink compositions can be supplemented through the additional of auxiliary proteins and other molecules such as growth factors including extracellular matrix components, Laminins, super affinity growth factors and morphogens. The bioink compositions can be used under physiological conditions related to 3D bioprinting parameters which are cytocompatible (e.g. temperature, printing pressure, nozzle size, bioink gelation process). The combination of a biogum-based biomaterial together with mammalian, plant, microbial or synthetically derived hydrogels exhibited improvement in printability, cell function and viability compared to tissues printed with bioink not containing biogums.

### CROSS-REFERENCE TO RELATED APPLICATIONS

This application relies on the disclosure of and claims priority to and the benefit of the filing date of U.S. Provisional Patent Application Nos. 62/750,390 and 62/750,417, filed Oct. 25, 2018, each of which is hereby incorporated by reference herein in its entirety.

### BACKGROUND OF THE INVENTION

### Field of the Invention

The present invention relates to the emerging fields of 3D bioprinting and functional tissue engineering. More specifically, embodiments of the invention relate to compositions which include biogums and/or botanical gums in combination with a biocompatible biomaterial to constitute a bioink capable of use in bioprinting of mammalian and human tissue constructs for subsequent use in in vitro culture, transplantation, tissue development, and drug screening and development.

### Description of Related Art

In three-dimensional (3D) printing processes, an object is fabricated layer by layer by a printer device using computer aided design, CAD file. 3D printing has been already successfully used in tissue engineering by many scientists to fabricate patient specific scaffolds. The scaffolds made of thermoplastic polymers have been extruded using 3D printers. The disadvantage of 3D printing using thermoplastic materials is a difficulty in cell seeding due to limited cell migration into porous structures. 3D Bioprinting operates using liquids in room or body temperature and thus can potentially handle living cells. The introduction of 3D Bioprinting is expected to revolutionize the field of tissue engineering and regenerative medicine, which can enable the reconstruction of living tissue and organs preferably using the patient's own cells. The 3D bioprinter is a robotic arm able to move in the X,Y,Z directions with a resolution of 10 μm while dispensing fluids. The 3D bioprinter can position several cell types and thus reconstruct the architecture of complex organs. The need for hierarchical assembly of 3D tissues has become increasingly important, considering that new technology is essential for advanced tissue fabrication. 3D cell printing has emerged as a powerful technology to recapitulate the microenvironment of native tissue, allowing for the precise deposition of multiple cells onto the pre-defined position. Parallel to these technological advances, the search for an appropriate bioink that can provide a suitable microenvironment supporting cellular activities has been in the spotlight. Bioinks often include a low viscosity or temperature sensitive biomaterial blended with a thickening agent to impart printability while also preserving cell viability and biological activity.

### SUMMARY OF THE INVENTION

According to embodiments, biogums such as microbially derived gums (e.g. xanthan gum(s)) or plant-derived (e.g., botanical) are utilized as a thickener in combination with various biomaterials to fabricate ready to print bioinks compatible with a range of printing nozzles and parameters. Embodiments of the invention rely on the discovery that the combination of two polymers, one a biomaterial-based hydrogel (mammalian, plant based, or microbially derived) or synthetic hydrogel and one a microbial, fungal, or plant based or produced biocompatible polysaccharide which acts as a thickener (e.g., xanthan gum, gellan gum, diutan gum, welan gum, pullalun gum, acacia gum, tara gum, glucomannan, pectin, locust bean gum, guar gum, carrageenan, and tragacanth), with or without cells, for use in the 3D bioprinting of human tissues and scaffolds, results in excellent printability and improved cell function, viability and engraftment.

Embodiments relate to a bioink composition which includes a biocompatible microbial (such as xanthan gum, gellan gum, curdlan gum, welan gum, pullalun gum), fungal, or plant-produced (such as acacia gum, tara gum, glucomannan, pectin, locust bean gum, guar gum, carrageenan, and tragacanth) polysaccharide, with or without cells, together with a mammalian, plant, microbial-derived, or synthetic hydrogel for bioprinting of human tissue analogues and scaffolds under physiological conditions. Furthermore, embodiments of the bioink composition can be supplemented through the addition of auxiliary proteins and other molecules such as extracellular matrix components, Laminins, growth factors including super affinity growth factors and morphogens. The bioink compositions can be used under physiological conditions related to 3D bioprinting parameters which are cytocompatible (e.g., temperature, printing pressure, nozzle size, bioink gelation process). According to one example, the combination of a microbial, fungal, or botanical biogum polysaccharide together with mammalian, plant, microbial or synthetically derived hydrogel exhibited improvement in printability, cell function and viability compared to tissues printed with bioink not containing these biogums. Embodiments thus include products (e.g., human tissue specific bioinks) and methods (e.g., physiological printing conditions), as well as several applications.

In a first aspect, provided is a bioink composition for use in 3D bioprinting comprising:

(i) one or more microbial, fungal, or plant based or produced thickener component (biogum and/or botanical gum),

(ii) one or more mammalian, plant, microbial, or synthetically derived biomaterial component, and

(iii) optionally, one or more auxiliary component,

wherein the bioink composition optionally includes cells.

In some embodiments the composition includes cells, such as human cells.

In some embodiments, the biogum is a xanthan gum produced from Gram negative bacteria of the Xanthomonas genus, including one or more of:

(i) X. campestris

(ii) X. fragaria 1822

(iii) X. arboricola

(iv) X. axonopodis

(v) X. citri

(vi) X. fragaria

(vii) X. gummisudans 2182

(viii) X. juglandis 411

(ix) X. phaseoli 1128

(x) X. vasculorium 702

In some embodiments, the biogum is a gellan gum produced from Gram negative bacteria Sphingomonas eldoda of the Sphingomonas genus.

In some embodiments, the biogum is a Curdlan gum produced from Gram negative bacteria of the Alcaligenes faecalis of the Alcaligenes genus.

In some embodiments, the biogum is a Welan gum produced from Gram negative bacteria of the Alcaligenes genus.

In some embodiments, the biogum is a Pullulan gum produced from the fungus Aureobasidium pullulans.

In some embodiments, the biogum is a botanical gum such as an acacia gum which is produced from plant species, including one or more of:

Acacia nilotica

Acacia Senegal

Vachellia (Acacia) seyal

Combretum, Albizia

In some embodiments, the biogum is a tara gum produced from T. spinos.

In embodiments, the biogum is a glucomannon produced from Amorphophallus konjac.

In embodiments, the biogum is a pectin from rinds of lemons, oranges, apples.

In embodiments, the biogum is a locust bean gum produced from Ceratonia siliqua.

In embodiments, the biogum is a guar gum produced from Cyamopsis tetragonolob.

In some embodiments, the biogum is a carrageenan produced from the Chondrus crispus (Irish moss).

In some embodiments, the biogum is a tragacanth produced from legumes of the genus Astragalus including one or more of:

A. adscendens

A. gummifer

A. brachycalyx

In some embodiments the ratio of xanthan gum or other microbial biogum, such as gellan gum, diutan gum, welan gum, or pullalun gum versus biomaterial by weight (w:w) is in the interval from 5:95 to 95:5 w:w, or from 80:20 to 20:80 w:w, such as 10:90, 20:80, 30:70, 40:60, 50:50, 60:40, 70:30, 80:20, 90:10 w:w, or any range encompassing or including these values.

In some embodiments, the xanthan gum or other microbial biogum, such as gellan gum, diutan gum, welan gum, or pullalun gum thickener component has a concentration in the interval from 0.5 to 20% weight by volume (w/v), including 0.6, 0.7, 0.8, 0.9, 1.0, 1.5, 2.0, 2.5, 3.0, 3.5, 4.0, 4.5, 5.0, 5.5, 6.0 6.5, 7.0, 7.5, 8.0, 8.5, 9.0, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% weight by volume (w/v), or any range encompassing or including these values such as 0.5 to 2% w/v, 2 to 5% w/v, 5 to 8% w/v, 8 to 10% w/v, 3 to 7.5% w/v, 1 to 6% w/v, 4 to 8% w/v, 5 to 15% w/v, 8 to 20% w/v, 2 to 18% w/v, and so on. This concentration level is relevant both as initial and final concentration, and after dilution with other components of the composition.

In some embodiments the ratio of botanical gums versus biomaterial by weight (w:w) is in the interval from 5:95 to 95:5 w:w, or from 80:20 to 20:80 w:w, such as 10:90, 20:80, 30:70, 40:60, 50:50, 60:40, 70:30, 80:20, 90:10 w:w, or any range encompassing/including these values.

In some embodiments, the botanical gums thickener component has a concentration in the interval from 0.5 to 50% weight by volume (w/v), including 0.6, 0.7, 0.8, 0.9, 1.0, 1.5, 2.0, 2.5, 3.0, 3.5, 4.0, 4.5, 5.0, 5.5, 6.0 6.5, 7.0, 7.5, 8.0, 8.5, 9.0, 9.5, 10, 12, 14, 16, 18, 20, 25, 30, 35, 40, 45% weight by volume (w/v), or any range encompassing or including these values such as 0.5 to 2% w/v, 0.4 to 1.2% w/v, 0.6 to 1.5% w/v, 2 to 5% w/v, 5 to 8% w/v, 8 to 10% w/v, 3 to 7.5% w/v, 1 to 6% w/v, 4 to 8% w/v, 5 to 50% w/v, 10 to 50% w/v, 10 to 40% w/v, 0.5 to 25% w/v, 20 to 50% w/v, 5 to 45% w/v, 1 to 10% w/v, 5 to 35% w/v, and so on. This concentration level is relevant both as initial and final concentration, and after dilution with other components of the composition.

In some embodiments, the mammalian, plant, microbial or synthetically derived biomaterial is chosen from at least one of the following constituents for cross-linking purposes and/or to contribute to rheological properties of the bioink, such as hydrocolloids or thickening and gelling agents: collagen type I, collagen and its derivatives, gelatin methacryloyl, gelatin and its derivatives, fibrinogen, thrombin, elastin, alginates (such as sodium alginate), agarose and its derivatives, glycosaminoglycans such as hyaluronic acid and its derivatives, chitosan, low and high methoxy pectin, biogums such as gellan gum, diutan gum, glucomannan gum, and/or carrageenans, nanofibrillated cellulose, microfibrillated cellulose, crystalline nanocellulose, carboxymethyl cellulose, methyl and hydroxypropylmethyl cellulose, bacterial nanocellulose, and/or any combination of these constituents.

In some embodiments the concentration of mammalian, plant, microbial or synthetically derived biomaterials is in the interval from 0.5 to 50% (w/v), such as 0.6, 0.7, 0.8, 0.9, 1.0, 1.5, 2.0, 2.5, 3.0, 3.5, 4.0, 4.5, 5.0, 5.5, 6.0 6.5, 7.0, 7.5, 8.0, 8.5, 9.0, 9.5, 10, 12, 14, 16, 18, 20, 25, 30, 35, 40, 45% (w/v), or any range encompassing or including these values, such as from 0.5 to 10% (w/v), and the concentration of cells is in the interval from 0.1 million/ml to 150 million/ml.

In some embodiments, the mammalian, plant, microbial or synthetically derived biomaterials include one or more of:

a. Alginate and its derivatives, such as sodium alginate

b. Agarose and its derivatives

c. Gelatin and its derivatives

d. Collagen and its derivatives

e. Fibrin and its derivatives

f. Hyaluronic acid

g. Basement membrane matrix

h. Laminins

i. Fibronectin and its derivatives

j. Heparan sulfate proteoglycans

k. Cellulose and its derivatives

l. Pectin and its derivatives

m. Chitosan and its derivatives

n. Silk and its derivatives, such as silk fibroin

o. Polyethylene glycol and its derivatives

p. Poly (vinyl alcohol)-based hydrogels

q. Poly(N-isopropylacrylamide) (PNIPAM)

r. Poly(2-hydroxypropyl methacrylate (PHPMA)

s. Poly(2-hydroxyethyl methacrylate) (PHEMA).

In some embodiments, the composition is provided under physiological conditions.

In some embodiments, the composition is provided so that at least one of the following conditions are met:

a. a pH-value for the composition in the interval from 5-8, including 5-7, or from 6-8, or from 7-8, or about 7;

b. the osmolarity of the composition is in the interval from 275 to 300 mOsm/kg, including 275-295, 280-295, 280-300, 285-300 mOsm/kg, such as about 295 mOsm/kg.

In some embodiments, the auxiliary components, such as biomaterials, may be in concentrations ranging from 0.5% to 50% w/v and may include one or more of:

a. Fibronectin and its derivatives

b. Collagen and its derivatives

c. Extracellular matrix

d. Basement membrane matrix

e. Fibrin and its derivatives

f. Elastin and its derivatives

g. Glycosaminoglycans and its derivatives including hyaluronic acid, Chondroitin sulfate, Dermatin sulfate, Heparin sulfate, Keratin sulfate

h. Laminin and its derivatives

i. Small Molecules

j. Peptides (Adhesive, Differentiation, Morphogenic)

k. Lysozyme

l. Growth factors and Morphogens including (a) Proliferative, (b) Differentiation (e.g., Chondrogenic, Fibrogenic, Myogenic, Cardiomyogenic, Neurogenic, Heptagenic, Pancreatic, Renal, Intestine, Dermal, Osteogenic, Oncogenic), and/or (c) Sternness Maintenance (e.g., Chondrogenic, Fibrogenic, Myogenic, Cardiomyogenic, Neurogenic, Heptagenic, Pancreatic, Renal, Intestine, Dermal, Osteogenic, Oncogenic)

m. Fluorescently labeled proteins and biomolecules.

In a second aspect, the invention relates to a method for 3D bioprinting of human tissue comprising bioprinting the composition of the invention, thereby combining a biogum (e.g., microbial gum, botanical gum), and a biomaterial derived from mammalian, plant, microbial or synthetic sources, with human or mammalian cells.

In a third aspect, the invention relates to a method for 3D bioprinting of at least one scaffold comprising bioprinting the composition of the invention, thereby combining a biogum (e.g., microbial gum, botanical gum) based thickener and a mammalian, plant, microbial or synthetic derived biomaterial.

In some embodiments, the method(s) for bioprinting of the invention is/are performed under physiological conditions.

In some embodiments related to the methods for bioprinting of the invention at least one of the following conditions are met during 3D bioprinting:

a. the temperature during the 3D bioprinting is in the interval from 4° C. to 40° C., including 10° C. to 40° C., 20° C. to 40° C., and 30° C. to 40° C., such as 37° C.; or

b. the printing pressure during the 3D bioprinting is in the interval from 1 to 200 kPa, such as below 50 kPa, including 5-45 kPa, 10-35 kPa, and 5-40 kPa, or in the interval from 5-25 kPa when bioprinting with cells.

In a further aspect, the invention relates to a bioprinted tissue or organ prepared by the method for 3D bioprinting with human cells according to invention.

In yet another aspect, the invention relates to the bioprinted tissue or organ according to the invention, for use in therapeutic applications including treatment of liver diseases, metabolic diseases, diabetes, heart diseases, kidney diseases, skin defects, bone defects, bone and soft tissue sarcomas, lung diseases, vessels repair, intestinal diseases, retinal defects, bladder diseases, prostate diseases, tissue fibrosis (e.g., liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue.

In another aspect, the invention relates to a method for treating liver diseases, metabolic diseases, diabetes, heart diseases, kidney diseases, skin defects, bone defects, bone and soft tissue sarcomas, lung diseases, vessels repair, intestinal diseases, retinal defects, bladder diseases, prostate diseases, tissue fibrosis (e.g., liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue comprising using the bioprinted tissue or organ according to the invention.

In still another aspect, the invention relates to a method for culturing the bioprinted tissue or organ of the invention, wherein the bioprinted tissue or organ is cultured under physiological or pathological conditions.

In some embodiments, at least two types of cells are co-cultured at different ratios. Ratios for cells in co-culture are chosen from: 1:1; 1:5, 1:10, 1:25, 1:50; 1:100, 1:150 and any range in between. In case of more than two cell types in culture the ratio is chosen from: 1:1:1; 1:1:5; 1:1:10; 1:1:50; 1:1:100 and any range in between.

In another embodiment, the method of culturing is for the purpose of in vitro culture, disease modelling, drug screening, biomarker discovery, tissue models for drug development, substance testing and bioactive compound efficacy testing.

In a further aspect the invention relates to an in vitro culture prepared by the method for culturing according to the invention.

The invention also relates to the use of the in vitro culture according to the invention for tissue development, disease development, drug screening and development and biomarkers.

In yet another aspect the invention relates to a bioprinted scaffold prepared by the method for 3D bioprinting according to the invention.

In still another aspect, the invention relates to the use of the bioprinted scaffold according the invention for wound healing.

In a further aspect, the invention relates to a method for preparing recellularised tissue, comprising repopulating the bioprinted scaffold of the invention.

In another aspect the invention relates to a recellularised bioprinted tissue, produced by repopulating the bioprinted scaffold of the invention with human cells.

In yet another aspect the invention relates to a bioprinted tissue, scaffold or recellularised bioprinted tissue of the invention, further comprising growth factors.

In still another aspect, the invention relates to a method for promoting tissue repair, comprising implanting the bioprinted tissue, scaffold or recellularised tissue comprising growth factors of the invention in a diseased tissue or organ.

In another aspect, the invention relates to a method of transplanting a bioprinted tissue, organ or scaffold of the invention, wherein the bioprinted scaffolds and/or tissues are implanted into the diseased tissue or organ, such as ectopically implanted subcutaneously or intra-omentum or directly as tissue-patches into the diseased tissue or organ.

In still another aspect, the invention relates to a method of repairing a tissue or an organ, wherein the bioprinted scaffolds and/or tissues of the invention are implanted as tissue-patches for improving wound healing.

In another aspect, the invention relates to a method of treating a disease in a tissue or an organ, wherein a bioprinted tissue of the invention or a recellularised bioprinted tissue of the invention is applied to the tissue or organ, such as by injection, implantation, encapsulation or extracorporeal application.

In one further aspect, the invention relates to a method for disease modelling, comprising the steps of:

a. providing a bioprinted scaffold of the invention;

b. performing mechanistic investigations; and/or

c. determining the effect of a compound, drug, biological agent, device or therapeutic intervention on the scaffold or tissue.

In still another aspect, the invention relates to a bioprinted tissue, scaffold or recellularised bioprinted tissue for use in one or more of:

a. implantation in a diseased tissue or organ;

b. transplantation into a human or animal body, whereby the bioprinted scaffolds and/or tissues are ectopically implanted subcutaneously or intra-omentum or directly as tissue-patches into the diseased tissue or organ;

c. repairing a tissue or an organ, whereby the bioprinted scaffolds and/or tissues are implanted as tissue-patches for improving wound healing; and/or

d. treating a disease in a tissue or an organ, wherein a bioprinted tissue of claim or a recellularised bioprinted tissue is applied to the tissue or organ, such as by injection, implantation, encapsulation or extracorporeal application.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is a graph showing temperature sweep for GelXG ranging from 33° C. to 15° C.; the plotted values are an average of two replicates. The storage modulus (G′) and loss modulus (G″) corresponds to the primary, left, axis while the tan 5 corresponds to the secondary, right axis.

FIG. 2 is a graph showing flow sweep of GelXG at four different temperatures, at shear rates between 0.002 s⁻¹ and 500 s⁻¹.

FIG. 3 is a graph showing frequency sweep of UV cross-linked GelXG at 20° C.; storage modulus and loss modulus correspond to the left axis and the complex viscosity corresponds to the right axis.

FIG. 4 is a graph showing amplitude sweep of UV cross-linked GelXG at 20° C. in the linear region of the frequency sweep performed prior to this test on the same sample.

FIG. 5 is a graph showing a temperature sweep for SilkInk.

FIG. 6 is a graph showing a flow sweep for SilkInk measured at 25° C.

FIG. 7 is a graph showing a flow sweep for SilkInk measured at different temperatures.

FIG. 8 is a graph showing frequency sweeps of SilkInk bioinks (cross-linked and not cross-linked) at 37° C.

FIGS. 9A-B are photographs showing one layer grid structures (FIG. 9A) printed with G3 bioink (chitosan-glucomannan bioink) and showing their calculated filament widths with varying speeds (FIG. 9B).

FIGS. 10A and 10B are photographs showing the chitosan-glucomannan bioink multi-layered constructs are stable.

FIG. 11 is a photograph showing the chitosan-glucomannan bioink provides a strong filament.

FIGS. 12A and 12B are graphs showing temperature sweeps for chitosan-glucomannan bioink G3 sample (FIG. 12A) and multiple samples (FIG. 12B).

FIGS. 13A and 13B are graphs showing flow sweeps for chitosan-glucomannan bioink at 25° C. (FIG. 13A) and 37° C. (FIG. 13B).

FIGS. 14A and 14B are graphs showing compressive stress-strain curves of different chitosan-glucomannan bioinks at 15 min (FIG. 14A) and 16 h (FIG. 14B) after crosslinking.

FIG. 15 is a table showing mechanical properties for chitosan and chitosan-glucomannan bioinks.

### DETAILED DESCRIPTION OF VARIOUS EMBODIMENTS OF THE INVENTION

Definition of terms and claim features.

“Biogum” refers to polysaccharides produced by a living organism such as bacteria or other microbials, fungi, or plants; examples of microbial biogums include xanthan gum, gellan gum, diutan gum, welan gum, and pullalun gum.

“Xanthan Gum” refers to a heteropolysaccharide with a primary structure that consists of pentasaccharide units consisting of two mannose, one glucuronic acid, and 2 glucose units. Xanthan consists of a backbone of glucose units with trisaccharide sidechains consisting of Mannose-Glucuronic Acid-Mannose linked to every other glucose unit at the 0-3 position.

“Gellan Gum” refers to a heteropolysaccharide with a primary structure that consists of tetrasaccharide units that consist of two glucose, one glucuronic acid, and one rhamnose unit. The backbone structure is glucose-gluruonic acid-glucose-rhamnose.

“Diutan Gum” refers to a polysaccharide consisting of a repeating unit that is composed of a six sugars. The backbone is made up of d-glucose, d-glucuronic acid, d-glucose, and 1-rhamnose, and the side chain of two 1-rhamnose.

“Welan Gum” refers consists of repeating tetrasaccharide units with single branches of L-mannose or L-rhamnose.

“Pullalun Gum” refers to a neutral polymer composed of α-(1,6)-linked maltotriose residues, which in turn are composed of three glucose molecules connected to each other by an α-(1,4) glycosidic bond.

“Botanical gum” refers to polysaccharide biogums isolated from plants; examples of botanical gums include acacia gum, tara gum, glucomannan, pectin, locust bean gum, guar gum, carrageenan, and tragacanth.

“Acacia Gum” refers to a heteropolysaccharide obtained from the Senegalia (Acacia) senegal and Vachellia (Acacia) seyal trees. This gum contains arabinogalactan which consists of arabinose and galactose monosaccharides that are attached to proteins creating what is known as arabinogalactan proteins.

“Tara Gum” refers to a heteropolysaccharide isolated from T. spinos of the Tara family consisting of a linear main chain of (1-4)-β-D-mannopyranose units attached by (1-6) linkages with α-D-galactopyranose units.

“Glucomannon” refers to a straight-chain polymer, with a small amount of branching isolated from the roots of the konjac plant. The component sugars are β-(1→4)-linked D-mannose and D-glucose in a ratio of 1.6:1.

“Pectin” refers to a heteropolysaccharide found in the primary cell walls of terrestrial plants. These include homogalacturonans are linear chains of α-(1-4)-linked D-galacturonic acid, rhamnogalacturonan II (RG-II), which is a complex and highly branched polysaccharide, amidated pectin, high-ester pectin, and low-ester pectin.

“Locust bean gum” refers to high-molecular-weight hydrocolloidal polysaccharides, composed of galactose and mannose units combined through glycosidic linkages, which may be described chemically as galactomannan. Locust bean gum is dispersible in either hot or cold water, forming a sol having a pH between 5.4 and 7.0, which may be converted to a gel by the addition of small amounts of sodium borate. Locust bean gum is composed of a straight backbone chain of D-mannopyranose units with a side-branching unit of D-galactopyranose having an average of one D-galactopyranose unit branch on every fourth D-mannopyranose unit.

“Guar gum” refers to an exo-polysaccharide composed of the sugars galactose and mannose. The backbone is a linear chain of β 1,4-linked mannose residues to which galactose residues are 1,6-linked at every second mannose, forming short side-branches.

“Carrageenan” refers to a polysaccharides isolated from red algae; carrageenan are high-molecular-weight polysaccharides made up of repeating galactose units and 3,6 anhydrogalactose (3,6-AG), both sulfated and nonsulfated. The units are joined by alternating α-1,3 and β-1,4 glycosidic linkages. Three classes of Carrageenan are Kappa, Iota, and Lambda. Kappa forms stiff gels in the presence of potassium and is isolated from Kappaphycus alvarezii. Iota forms soft gels in the presence of calcium ions and is isolated from Eucheuma denticulatum. Lambda does not gel, and is used as a pure thickener.

“Tragacanth” refers to a dried sap of several species of Middle Eastern legumes of the genus Astragalus, including A. adscendens, A. gummifer, and A. brachycalyx.

“Mammalian, plant, microbial, or synthetic hydrogels” refers to any biocompatible polymer network that exhibits characteristics of a hydrogel. A hydrogel is a polymer network that has hydrophilic (e.g., water binding) properties. Mammalian hydrogels consist of proteins or polymers derived from the various tissues, organs, and cells found in mammals including humans, porcine, bovine. Plant hydrogels consist of proteins or polymers derived from various plants including trees, algae, kelp, seaweed. Microbial hydrogels (also referred to as biogums) include polysaccharides produced by bacteria such as xanthan gum, gellan gum, diutan gum, welan gum, and pullalun gum. Synthetic hydrogels include polymers derived from polyethylene, polyethylene, polycaprolactone, polylactic, polyglycolic acid, and their derivatives.

“Bioprinting” refers to the utilization of 3D printing and 3D printing-like techniques to combine cells, growth factors, and biomaterials to fabricate biomedical parts that maximally imitate natural tissue characteristics. Generally, 3D bioprinting utilizes the layer-by-layer method to deposit materials known as bioinks to create tissue-like structures that are later used in medical and tissue engineering fields.

As used herein, “physiological conditions” include conditions (such as pH, osmolarity, temperature and printing/extrusion pressure) that are typical to the normal living environment for a culture or cells, such as, for human cells, a temperature around 37° C., such as in the interval from 35-39° C., a printing pressure in the interval from 1 kPa to 200 kPa, such as below 25 kPa, a pH in the interval from 5-8, such as about 7, and an osmolarity in the interval from 275 to 300 mOsm/kg, such as about 295 mOsm/kg.

As used herein, “pathological conditions” include exposure of a culture or cells to inflammatory and/or carcinogenic conditions, e.g. recapitulating the disease.

As used herein, “co-culturing” cells means that cells of at least two types are cultured together.

In the context of the present invention, the term “bioprinted scaffold” refers to a bioprinted structure or tissue printed with a composition without cells. On the other hand, the term “bioprinted tissue” refers to a bioprinted structure or tissue printed with a composition with cells. The cells can be autologous, allogeneic or xenogeneic. The cells can be stem cells (e.g., pluripotent, induced pluripotent, multipotent, totipotent; mesenchymal, hematopoietic, embryonic, umbilical cord), primary cells (e.g., primary hepatocytes, primary renal cells), or immortalized cells. The cells can be or include can be or include, for example, cells from tissues such as liver, kidney, heart, lung, gastrointestinal, muscle, skin, bone, cartilage, vascularized tissues, blood vessels, ducts, ear, nose, esophagus, trachea, and eye. These can include endothelial cells, skin cells such as keratinocytes, melanocytes, Langerhans' cells, and Merkel cells, connective tissue cells such as fibroblasts, mast cells, plasma cells, macrophages, adipocytes, and leukocytes, bone tissue cells such as osteoblasts, osteoclasts, osteocytes, and osteoprogenitor (or osteogenic) cells, cartilage cells such as chondrocytes and chondroblasts, muscle cells such as smooth muscle cells, skeletal muscle cells, cardiac muscle cells, any cells having muscle fibers such as type I (slow twitch), type IIa and type IIb (fast twitch), nerve cells such as multipolar neurons, bipolar neurons, unipolar neurons, sensory neurons, interneurons, motor neurons, neurons of the brain (e.g. Golgi cells, Purkinje cells, pyramidal cells), glial cells such as oligodendrocytes, astrocytes, ependymal cells, Schwann cells, microglia, and satellite cells, liver cells such as hepatocytes, biliary epithelial cells (cholangiocytes), stellate cells, Kupffer cells, and liver sinusoidal endothelial cells, kidney cells such as glomerulus parietal cells, glomerulus podocytes, proximal tubule brush border cells, Loop of Henle thin segment cells, thick ascending limb cells, kidney distal tubule cells, collecting duct principal cells, collecting duct intercalated cells, and interstitial kidney cells, pancreatic cells such as islets cells, alpha cells, beta cells, delta cells, PP cells, endocrine gland cells such as pancreatic cells, hypothalamus cells, pituitary cells, thyroid cells, parathyroid cells, adrenal cells, pineal body cells, and ovarian cells and testicular cells, exocrine gland cells such as sweat gland cells, salivary gland cells, mammary gland cells, ceruminous gland cells, lacrimal gland cells, sebaceous gland cells, and mucous gland cells, or epithelial cells such as squamous cells, cuboidal cells, and columnar cells arranged in architectures such as simple, stratified, and pseudostratified.

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Composition

In a first aspect the invention relates to a bioink composition comprising a biogum-based thickener, and a mammalian, plant, microbial or synthetic derived biomaterial with or without cells depending on the application, with or without auxiliary components.

In embodiments, bioink compositions of the invention can comprise one or more biogum thickener, one or more mammalian, plant, microbial or synthetic biomaterial, and one or more auxiliary components.

The biogums can be derived from different mechanical, enzymatic and/or chemical steps known in the art which are performed on the source material (e.g., plant based (or botanical), fungal, or microbial). The bioink compositions or components are typically prepared using sterile components and prepared in clean room conditions.

In embodiments, the bioink composition can include one or more buffer such as HEPES (4-(2-hydroxyethyl)-1-piperazineethanesulfonic acid), PIPES (piperazine-N,N′-bi s(2-ethanesulfonic acid)), TES (2-[(2-Hydroxy-1,1-bis(hydroxymethyl)ethyl)amino] ethane sulfonic acid, N-[Tris(hydroxymethyl)methyl]-2-aminoethanesulfonic acid), and CAPS (N-cyclohexyl-3-aminopropanesulfonic acid). The bioink composition can also include one or more solvent such as distilled water, saline, or pH buffered saline. The osmolarity of the composition can be designed to provide compatibility with one or more cell types.

In some embodiments, the composition or one or more of its individual components are provided in desiccated form suitable for reconstitution with a solvent or buffering agent.

Methods for Bioprinting

In another aspect, the invention relates to methods for preparing bioprinted tissues or scaffolds that are suitable for use in the various products, uses and methods of the invention.

In general, the method for 3D bioprinting of human tissue (with cells) or scaffolds (without cells) comprises combining one or more biogum-based bioink, (with or without human cells), and human tissue-specific extracellular matrix (ECM) material, wherein the 3D bioprinting is performed under physiological conditions.

The 3D bioprinted tissue or scaffold can be in the form of a grid, drop, tissue-specific shapes like hepatic lobule for liver etc., or the like. The 3D bioprinted tissue, construct or scaffold can have a printed size in the interval from 0.1 mm to 50 cm in diameter and/or length or width. The bioprinter apparatus can be of any commercially available type, such as the 3D Bioprinters' INKREDIBLE™, INKREDIBLE+™ or BIO X™ from CELLINK AB, or any conventional robotic bioprinter having standard components such as motors, print heads, print bed, substrates for printing, printed structures, cartridges, syringes, platforms, lasers and controls.

Kits for Bioprinting

In one embodiment, the bioink composition is provided in a kit comprising the composition loaded into one or more cartridges, vials, or syringes. The composition can be provided in desiccated form in the kit. The kit can include a separate buffer or solvent for reconstituting the composition, or the composition can be provided already reconstituted with the buffer or solvent already contained in the same cartridge, vial, or syringe as the composition.

A method for preparing bioprinted tissues or scaffolds can be performed under physiological conditions, which could vary depending on the tissue and/or the cells that are printed. Typically, the conditions and parameters during bioprinting varies within the following intervals:

Temperature: 4° C. to 40° C.

Printing pressure: 1-200 kPa.

Also, external cross-linking may be used during or after the bioprinting process such as calcium chloride solution, UV or light exposure in the wavelengths between 300 and 800 nm, such as 365 nm, 405 nm, 425 nm, and 480 nm, or self-assembly of the biomaterial component under thermal incubation. Photoinitiators that can be used include lithium phenyl-2,4,6-trimethylbenzoylphosphinate or LAP. Other photoinitiators can include free radical photoinitiators, cationic photoinitiators, and anionic photoinitiators. The photoinitiator forms a free radical, cation, or anion which subsequently reacts and catalyzes a polymerization or cross-linking reaction. Examples of other photoinitiators include, but are not limited to benzophenone, benzoin-ether, 2-(dimethylamino)ethanol (DMAE), hydroxyacetophenones, 2-hydroxy-2-methyl-1-phenylpropan-1-one and, hydroxyl-phenyl-ketone, Irgacure® 2959, 2-hydroxy-4′-(2-hydroxyethoxy)-2-methylpropiophenone, (2-hydroxy-1-[4-(2-hydroxyethoxy) phenyl]-2-m ethyl-1-propanone; (2,2′-azobis[2-methyl-N-(2-hydroxyethyl)propionamide]; 2-isocyanotoethyl methacrylate; benzoyl benzylamine; camphorquinone; thiol-norbornene (thiol-ene); riboflavin; lucirin-TPO; Rose Bengal/furfuryl; ethyl eosin; methacrylic anhydride; 2,2-dimethoxy-2-phenylacetophenone; and Eosin Y.

Bioprinted Tissue or Organ

Other aspects of the invention provide a bioprinted tissue produced by a method described above. Bioprinted tissues produced as described herein display the tissue-specific extracellular matrix protein composition of the source tissue sample.

Bioprinted Scaffold+Use of Bioprinted Scaffold

Another aspect of the invention provides a bioprinted human scaffold or tissue produced as described above for the use in tissue repair, for example.

Bioprinted scaffolds with or without cells and/or with or without known growth factors can be implanted in diseased-tissues or organs, such as tissue-patches, in order to promote tissue repair. For instance, tissue repair can be promoted by wound healing due to the capability of ECM to favor immunomodulation and therefore reducing tissue scarring in fibrotic diseases (e.g. liver fibrosis, intestinal fibrosis, fistulas, Chron's Disease, cartilage defects, etc.).

Methods for Culturing Bioprinted Tissue+In Vitro Culture+Use of In Vitro Culture

Embodiments of the invention provide a bioprinted human scaffold or tissue produced as described above for the use in modeling human diseases, testing drugs and biomarker discovery.

Bioprinted tissue can be used to screen drugs and/or cell-based therapies. For example, bioprinted tissue with cancer cells can be exposed to chemotherapy agents, immunotherapy and/or CAR-T, NK cells.

Methods of Transplantation

Yet another aspect of the invention provides a bioprinted human scaffold or bioprinted human tissue produced as described above for use in the transplantation of a tissue or organ in an individual.

For example, a bioprinted human scaffold or bioprinted human tissue may be transplanted to an individual to replace an organ or a tissue.

Methods of Repairing or Regenerating Tissues and Organs

Another aspect of the invention provides a bioprinted human scaffold or bioprinted human tissue produced as described above for use in the treatment of disease or dysfunction in a tissue or organ in an individual.

For example, a bioprinted human scaffold or bioprinted human tissue may be implanted in an individual to regenerate a complete new organ or to improve the repair of a damaged organ, or may support the organ function of the individual from outside the body.

Methods of Treating a Disease

The bioprinted scaffold or tissue may be useful in therapy, for example for the replacement or supplementation of tissue in an individual.

A method of treatment of a disease may comprise implanting a bioprinted human scaffold or bioprinted human tissue produced as described above into an individual in need thereof.

The implanted bioprinted scaffold or tissue may replace or supplement the existing tissue in the individual.

The bioprinted scaffold or tissue may be used for the treatment of any one of the diseases chosen from, but not limited to: liver diseases, metabolic diseases, diabetes, heart diseases, kidney diseases, lung disease, skin defects, muscle defects, bone defects, bone and soft tissue sarcomas, lung diseases, vessels repair, intestinal diseases, fistulas, cartilage defects, retinal defects, bladder diseases, prostate diseases, tissue fibrosis (e.g. liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue disclosed in this application, comprising using the bioprinted tissue, organ or scaffold.

Methods for Disease Modelling

The bioprinted tissue or bioprinted scaffold may be useful for disease modelling. Suitable ECM source(s) may be derived from a normal tissue sample or pathological tissue sample, as described above.

A method of disease modelling may comprise:

providing a bioprinted tissue or scaffold produced as described above, optionally bioprinting the tissue or scaffold with cells to produce a recellularised bioprinted tissue, and determining the effect of a compound, drug, biological agent, device or therapeutic intervention on the bioprinted scaffold or tissue or the cells therein.

Methods described herein may be useful in modelling tissue diseases or diseases affecting the tissue, such as tissue fibrosis, tissue cancer and metastases, tissue drug toxicity, post-transplant immune responses, and autoimmune diseases.

Methods of Diagnosis

Bioprinted scaffolds and tissues may be useful for the diagnosis of disease. Suitable bioprinted scaffolds and tissues may be derived from tissue from an individual suspected of having a disease in the tissue or organ.

A method of diagnosing disease in a human individual may comprise: providing a bioprinted scaffold or tissues from the individual produced as described above, and determining the presence and amount of one or more scaffold proteins in the sample.

The presence and amount of scaffold proteins in the sample may be indicative of the presence of disease in the tissue or organ of the individual.

Other Applications

The bioprinted scaffolds and tissues may also be useful for proteomics, biomarker discovery, and diagnostic applications. For example, the effect of a protease on the components, architecture or morphology of a bioprinted scaffold and tissue may be useful in the identification of biomarkers.

The invention will now be described by the following non-limiting examples:

### EXAMPLES: GELXG, SILKINK, AND CHITOSAN-GLUCOMANNAN BIOINK

Examples 1-3 provide rheological data for a bioink composition of the invention (GelXG). The bioink composition GelXG comprises: 5% GelMA (Gelatin Methacryloyl)+1.5% Xanthan Gum+LAP 0.25% (lithium phenyl-2,4,6-trimethylbenzoylphosphinate)+2.3% mannitol+10 mM HEPES (4-(2-hydroxyethyl)-1-piperazineethanesulfonic acid) as a buffer. Example 4 provides rheological data for another bioink composition of the invention (SilkInk). The bioink composition SilkInk comprises: 15% w/v silk fibroin+1% w/v Alginate (e.g., sodium alginate)+10% w/v Xanthan Gum as thickener. Example 5 provides rheological data for another bioink composition of the invention (chitosan-glucomannan bioink), which bioink composition comprises 3.18% w/v chitosan, 1.818% w/v glycerol phosphate disodium salt (GP), and glucomannan (GM) in an amount of 0.909% w/v. These examples are merely illustrative and should not be construed as limiting any particular feature of the invention.

### Example 1: Temperature Dependence of Viscoelastic Properties (GelXG)

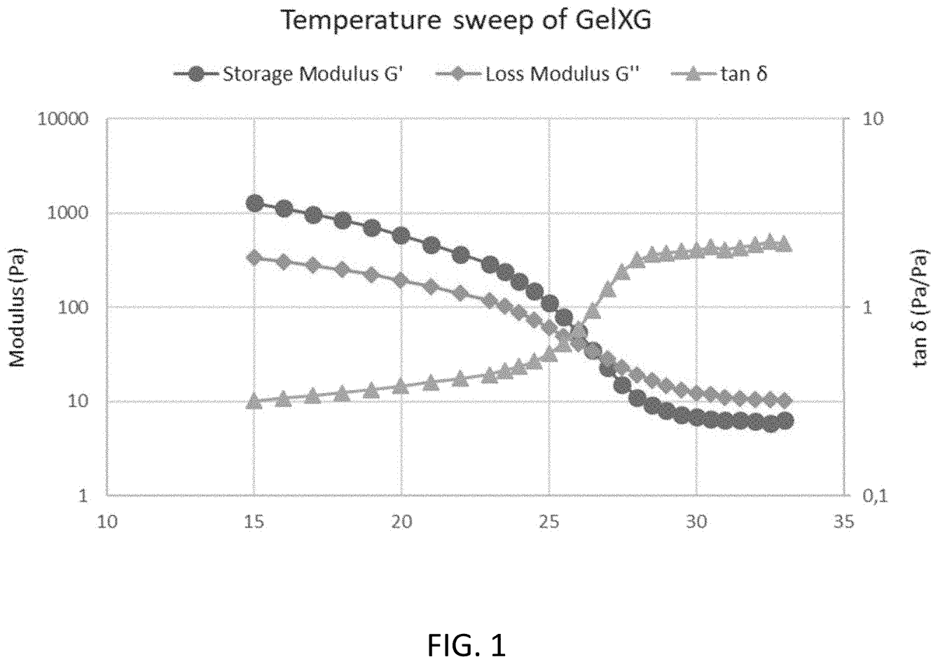

The test was performed using a 20 mm plate-plate geometry (Discovery Hybrid Rheometer 2, TA instruments, UK), starting at 33° C. and finishing at 15° C. The test is run at a constant angular frequency of 10 rad/s. Average values, from two replicates, of the storage modulus G′, loss modulus G″ and tan 8 are presented in FIG. 1.

### Example 2: Viscosity Analysis (GelXG)

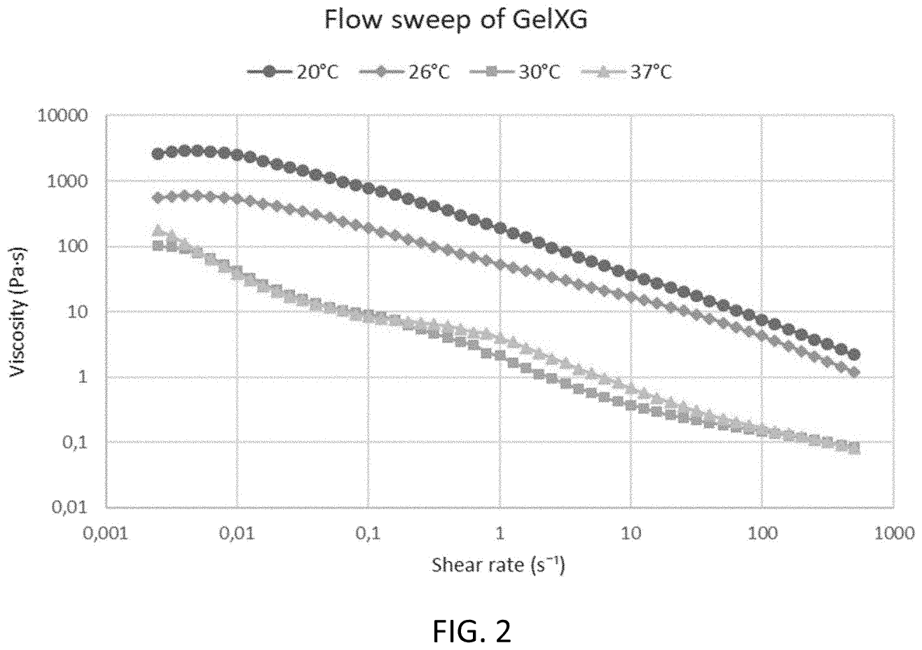

The test was performed using a 20 mm plate-plate geometry (Discovery Hybrid Rheometer 2, TA instruments, UK). The flow sweep was performed at four temperatures: 20° C., 26° C., 30° C. and 37° C., at shear rates ranging from 0.002 s−1 to 500 s 1. The flow sweeps are compared in FIG. 2.

### Example 3: Properties of Cross-Linked Samples (GelXG)

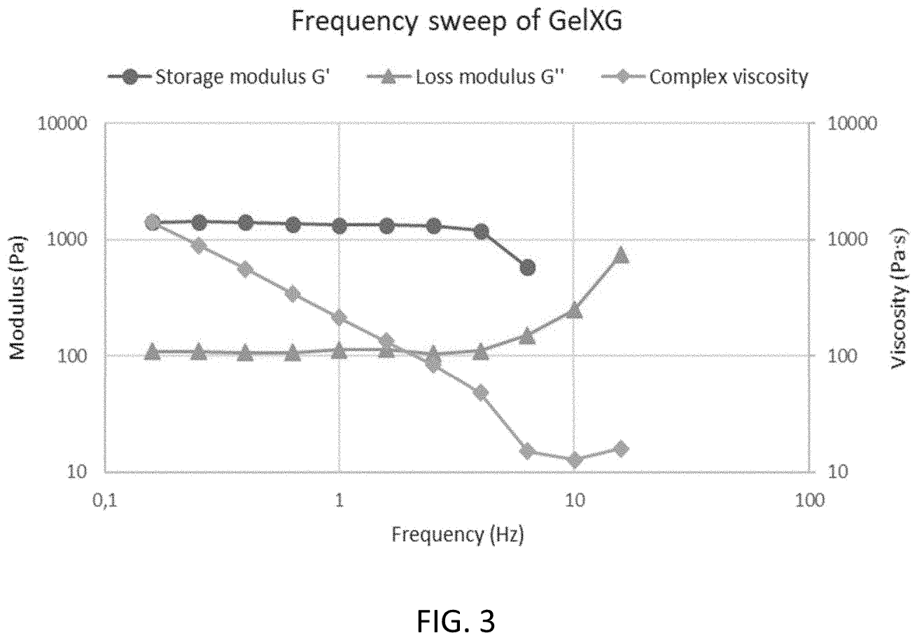

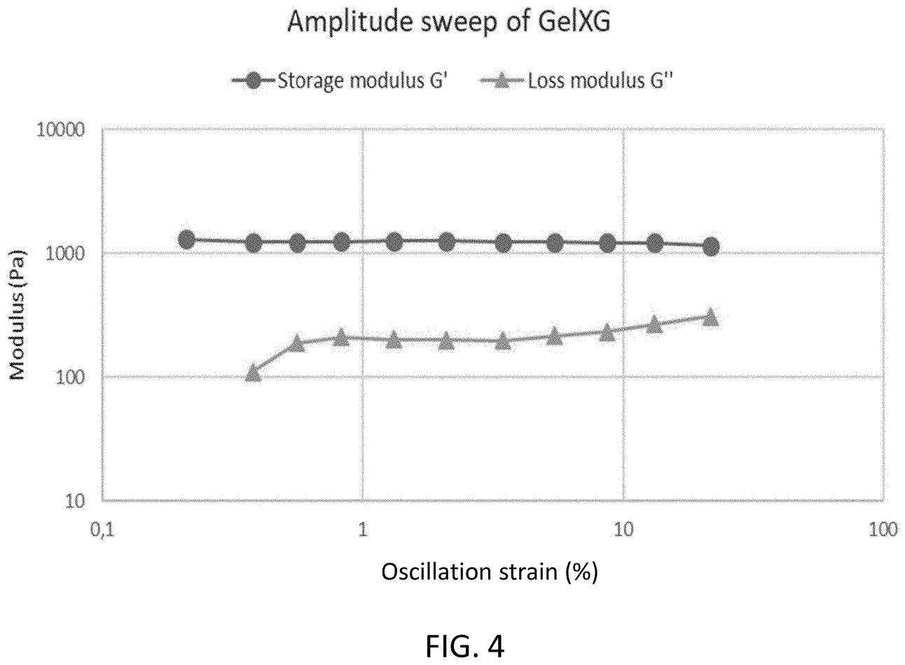

The tests were performed using an 8 mm serrated plate-plate geometry (Discovery Hybrid Rheometer 2, TA instruments, UK). A frequency sweep was performed between 0.16 Hz and 6.3 Hz, the storage modulus, loss modulus and complex viscosity were plotted. Thereafter, an amplitude sweep at a frequency from the linear region of the storage modulus was performed at the same sample. All tests were performed at 20° C., on 3D printed samples (diameter=8 mm, height=2 mm) which, had been cross-linked with UV (405 nm) for 20 s. FIG. 3 shows the results from the frequency sweep of UV cross-linked GelXG and FIG. 4 shows the results from the amplitude sweep of the same GelXG sample.

### Example 4 (SilkInk)

Preparation: A first solution (30% w/v silk fibroin (SF) solution) and a second solution (Alginate (e.g., sodium alginate) xanthan gum (XG) blend) were mixed together using a Luer lock adapter in 1:1 ratio between the syringes by moving them back and forth up to 10 times to result in the final concentration of components (15% w/v silk fibroin+1% w/v Alginate (sodium alginate)+10% w/v Xanthan Gum). Three batches were prepared in total. The first batch apparently had some silk self-assembly after mixing, while the other two were mixed even more gently to minimize this effect.

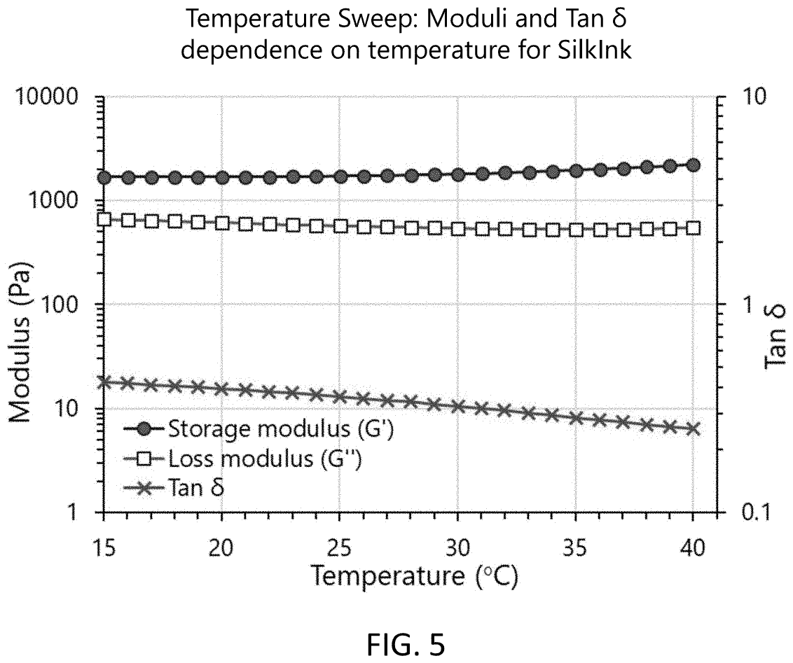

FIG. 5 is a graph showing a temperature sweep of SilkInk samples. The SilkInk bioink is not temperature sensitive, showing almost identical performance throughout the 15-40° C. range (there is a slight G′ increase above 30° C., might be due to the silk protein assembly). The SilkInk bioink has no distinct gel point as G′ is always higher than G″.

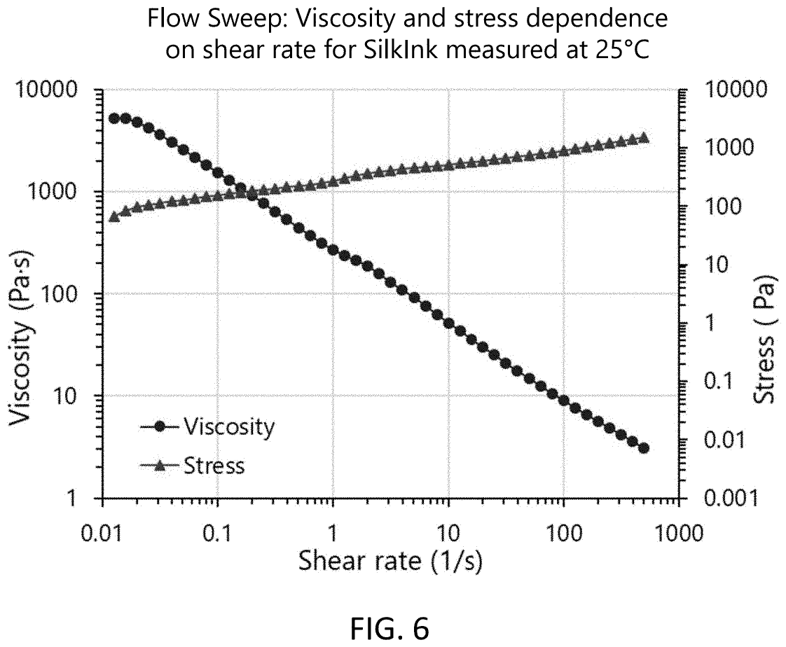

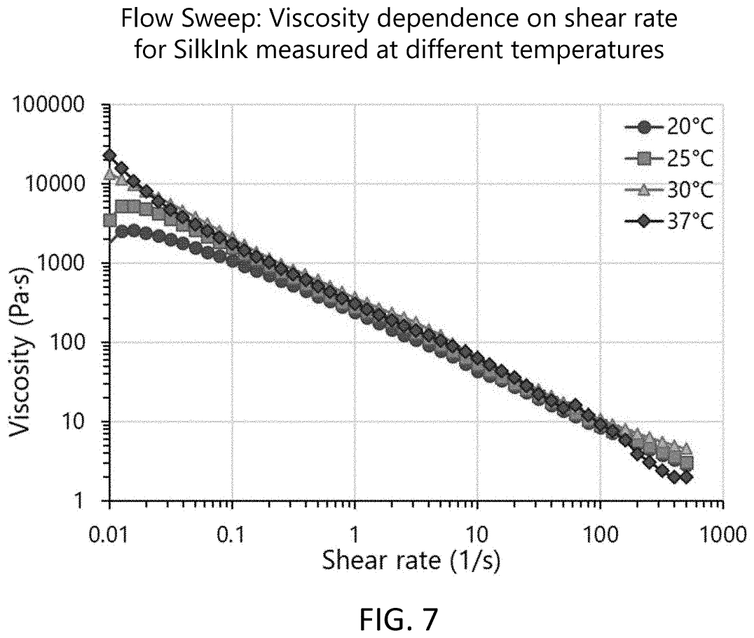

FIGS. 6 and 7 are flow sweeps showing extremely stable shear thinning behavior for the bioink at wide shear rate range (FIG. 6) and very similar shear thinning behavior for the bioink at different temperatures-confirming that SilkInk is a temperature-insensitive bioink (FIG. 7).

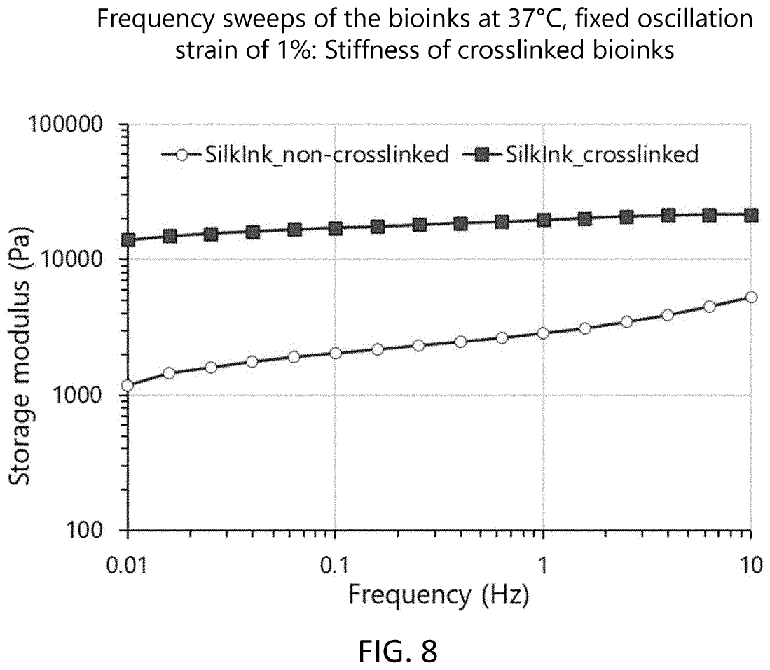

FIG. 8 is a graph showing frequency sweeps of SilkInk samples (cross-linked and non-cross-linked). For the crosslinked SilkInk, the storage modulus at 1 Hz is above 20 kPa, which is high enough to make the constructs robust. For the non-crosslinked SilkInk, the storage modulus is lower, though higher than 1 kPa; the non-crosslinked SilkInk should stay stable even without crosslinking, though handling might be more difficult. For the non-crosslinked SilkInk, the storage modulus increases with higher oscillation frequency, an indication of continuous silk self-assembly after extrusion.

### Example 5 (Chitosan-Glucomannan Bioink)

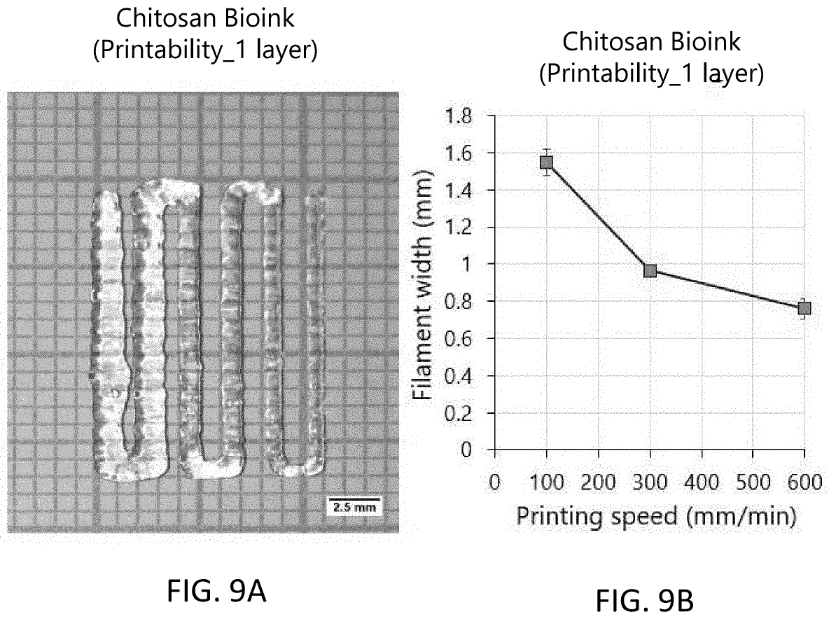

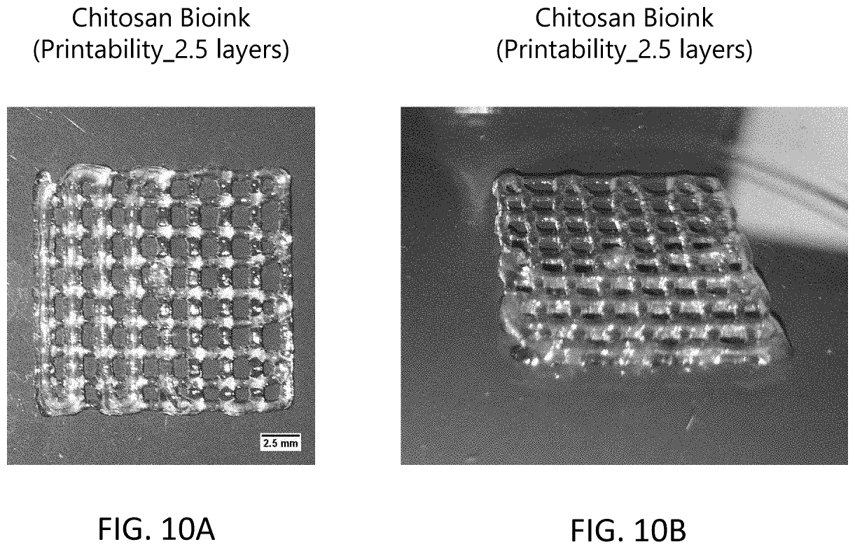

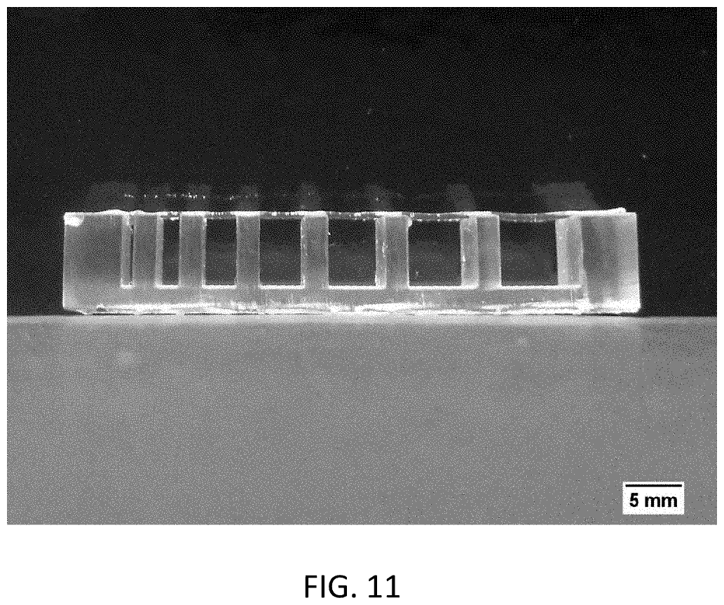

FIGS. 9A and 9B show one layer grid structures printed with G3 bioink (FIG. 9A) and their calculated filament widths with varying speeds (FIG. 9B). The chitosan-glucomannan bioink demonstrates good printability of 1 layer; no clogging, smooth lines, however higher concentrations of glucomannan result in clogging and filament breaks. FIGS. 10A and 10B are photographs showing the chitosan-glucomannan bioink multi-layered constructs are stable (e.g., smooth lines). FIG. 11 shows the chitosan-glucomannan bioink provides a strong filament-no signs of collapse even at 7 mm gap.

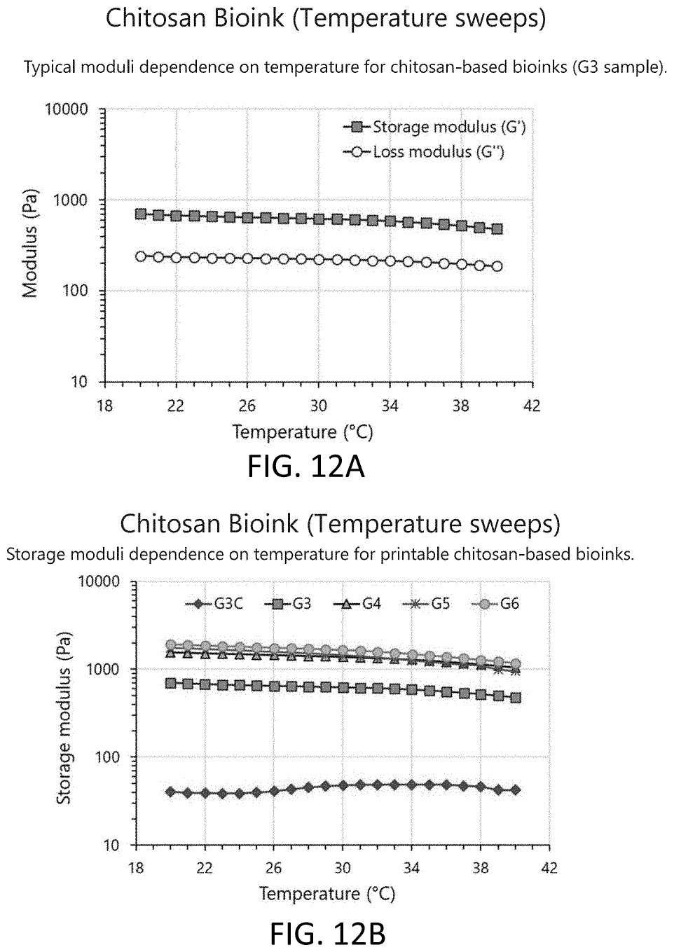

FIGS. 12A and 12B are temperature sweeps showing the chitosan bioink is completely temperature-independent after mixing with a crosslinking agent, which in this case is sodium tripolyphosphate (STPP) (FIG. 12A). For G3, storage modulus is around 0.6 kPa at RT (FIG. 12B). The addition of glucomannan is crucial for making the bioinks stiffer (G3 vs G3C). G4-6 contain more glucomannan than G3 and thus have a higher storage modulus than G3.

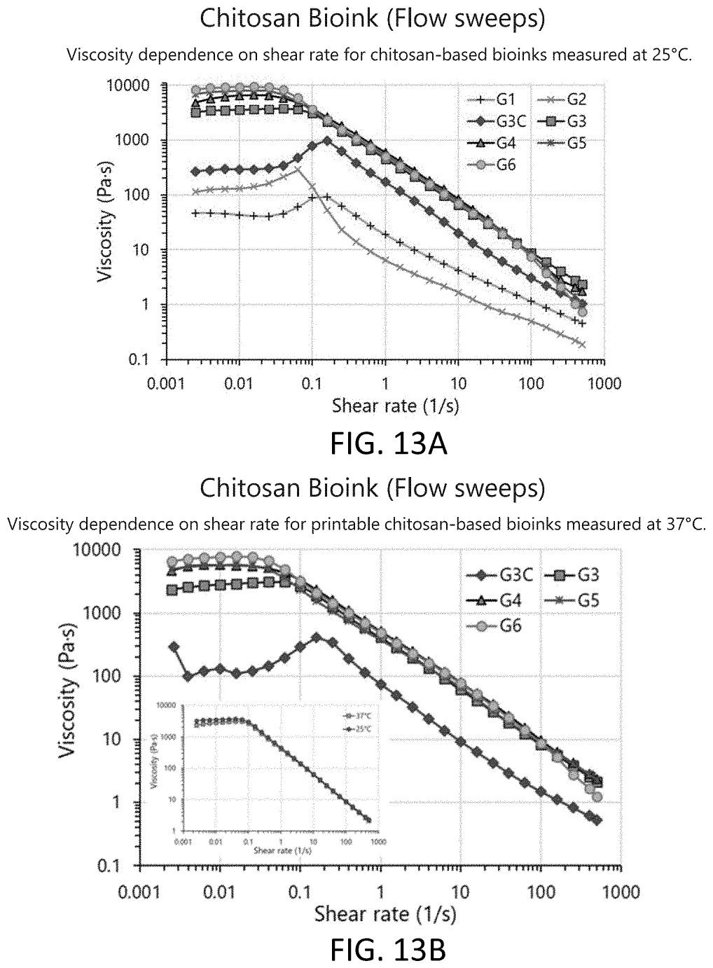

FIGS. 13A and 13B are flow sweeps showing the chitosan bioink exhibited good shear thinning behavior for all samples above the shear rate of 0.2 (FIG. 13A). The viscosity is proportional to chitosan/GP ratio (G1, G2 and G3C), however with the addition of glucomannan this relationship disappears-glucomannan determines viscosity. All glucomannan-containing bioinks show excellent shear thinning behavior even at 37° C. (FIG. 13B) (almost identical to 25° C., inset image).

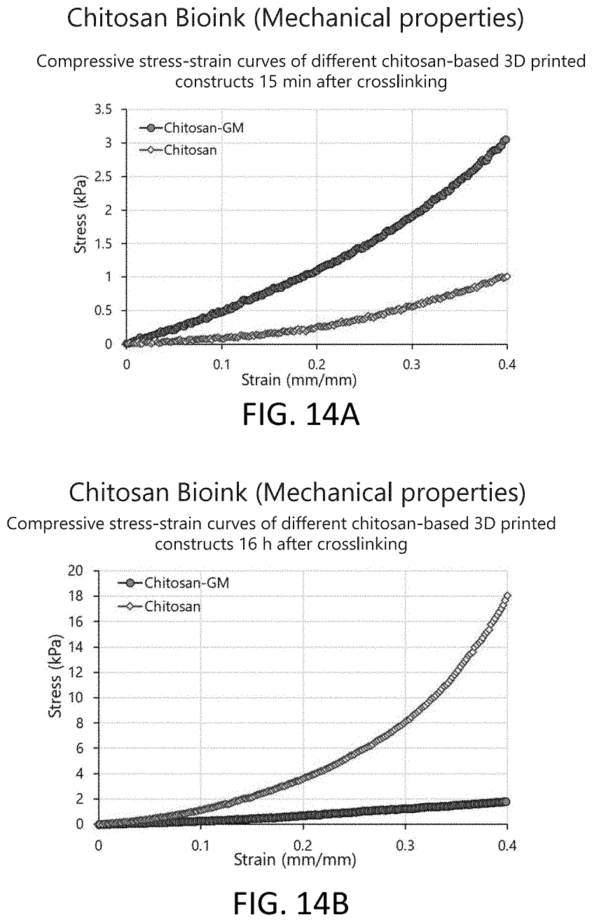

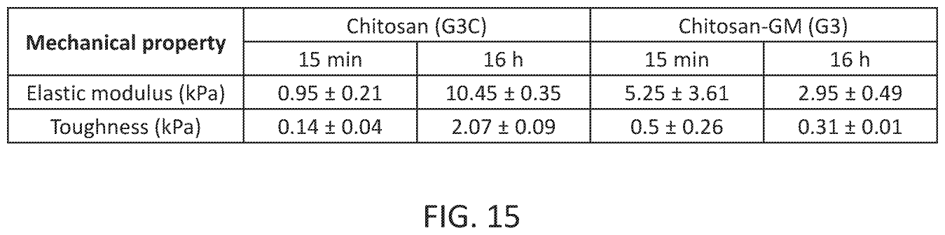

FIGS. 14A and 14B are compressive stress-strain curves of different chitosan-based 3D printed constructs 15 min (FIG. 14A) and 16 h (FIG. 14BB) after crosslinking. A 100N load cell UTS (Instron 5565A, UK) was used at a compression rate of 1%/s until 40% strain was reached. Cylindrical samples (d=8 mm, h=2 mm) were prepared in a gel caster, and elastic modulus and toughness were calculated (values shown in the table of FIG. 15).

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-11. (canceled)

12. A method for diagnosing a disease of a human individual comprising the steps of:
a. providing a sample from an individual of a bioprinted tissue or scaffold prepared by a method for 3D bioprinting of human or mammalian tissue comprising:
bioprinting a composition comprising one or more microbial, fungal, or plant based or produced biogum and one or more biomaterials derived from mammalian, plant, microbial or synthetically derived hydrogels in a manner that combines the one or more biogum and the one or more biomaterials with human or mammalian cells or in a manner that forms at least one scaffold;
b. determining the presence and amount of one or more scaffold or tissue proteins in the sample, wherein the presence and amount of the one or more scaffold or tissue proteins in the sample is indicative of the presence of diseases in the tissue or organ of the individual and can reflect response to treatment.

13. (canceled)

14. The method of claim 12, wherein:
a. a temperature during the 3D bioprinting is in the range of 4° C. to 40° C.; and/or
b. a printing pressure during the 3D bioprinting is in the range of 1 to 200 kPa.

15. A method comprising:
preparing a bioprinted tissue or organ by bioprinting a composition comprising one or more microbial, fungal, or plant based or produced biogum and one or more biomaterials derived from mammalian, plant, microbial or synthetically derived hydrogels, wherein the bioprinted tissue or organ is bioprinted in a manner that combines one or more of the microbial, fungal, or plant based or produced biogums and one or more of the biomaterials with human or mammalian cells;
culturing the bioprinted tissue or organ under physiological or pathological conditions to prepare an in vivo culture; and
performing tissue development, disease development, drug screening and development and biomarkers with the in vivo culture.

16. The method of claim 15, wherein the performing involves therapeutic applications of liver diseases, metabolic diseases, diabetes, heart diseases, kidney diseases, skin defects, muscle defects, bone defects, bone and soft tissue sarcomas, lung diseases, vessels repair, intestinal diseases, fistulas, cartilage defects, retinal defects, bladder diseases, prostate diseases, tissue fibrosis, or cancer.

17. The method of claim 15, wherein the performing involves treatment of liver diseases, metabolic diseases, diabetes, heart diseases, kidney diseases, skin defects, muscle defects, bone defects, bone and soft tissue sarcomas, lung diseases, vessels repair, intestinal diseases, fistulas, cartilage defects, retinal defects, bladder diseases, prostate diseases, tissue fibrosis, or cancer.

18. (canceled)

19. The method of claim 15, wherein the culturing comprises co-culturing at least two types of cells at different ratios chosen from: 1:1; 1:5, 1:10, 1:25, 1:50; 1:100, 1:150 and any range in between, or in case of more than two cell types in culture the ratio is chosen from: 1:1:1; 1:1:5; 1:1:10; 1:1:50; 1:1:100 and any range in between.

20. The method of claim 15, wherein the bioprinted tissue or organ is capable of one or more applications comprising in vitro culture, disease modelling, drug screening, biomarker discovery, tissue models for drug development, substance testing and bioactive compound efficacy testing.

21-35. (canceled)

36. The method of claim 12, wherein one or more of the biogums is a microbial gum comprising xanthan gum, gellan gum, diutan gum, welan gum, or pullalun gum, or combinations thereof.

37. The method of claim 12, wherein one or more of the biogums is a botanical gum comprising acacia gum, tara gum, glucomannan, pectin, locust bean gum, guar gum, carrageenan, or tragacanth, or combinations thereof.

38-42. (canceled)

43. The method of claim 12, wherein the composition comprises a ratio of one or more of the biogums to one or more of the biomaterials in a range of 5:95 to 95:5.

44. The method of claim 12, wherein one or more of the biomaterials of the composition comprises at least one hydrocolloid, thickening, or gelling agent capable of cross-linking and/or contributing to rheological properties of the composition.

45. The method of claim 12, wherein the composition comprises one or more additional biopolymers capable of cross-linking and/or contributing to rheological properties of the composition.

46. The method of claim 45, wherein the one or more additional biopolymers comprises one or more alginates, hyaluronic acid and its derivatives, agarose and its derivatives, chitosan, fibrin, gellan gum, silk and its derivatives, nanofibrillated cellulose, microfibrillated cellulose, crystalline nanocellulose, bacterial nanocellulose, carrageenans, elastin, collagen and its derivatives, gelatin and its derivatives, or any combination thereof.

47. The method of claim 12, wherein the human or mammalian cells are chosen from one or more of human, porcine, or bovine cells.

48. The method of claim 15, wherein the composition comprises a ratio of one or more of the biogums to one or more of the biomaterials in a range of 5:95 to 95:5.

49. The method of claim 15, wherein one or more of the biomaterials of the composition comprises at least one hydrocolloid, thickening, or gelling agent capable of cross-linking and/or contributing to rheological properties of the composition.

50. The method of claim 15, wherein the composition comprises one or more additional biopolymers capable of cross-linking and/or contributing to rheological properties of the composition.

51. The method of claim 50, wherein the one or more additional biopolymers comprises one or more alginates, hyaluronic acid and its derivatives, agarose and its derivatives, chitosan, fibrin, gellan gum, silk and its derivatives, nanofibrillated cellulose, microfibrillated cellulose, crystalline nanocellulose, bacterial nanocellulose, carrageenans, elastin, collagen and its derivatives, gelatin and its derivatives, or any combination thereof.

52. The method of claim 15, wherein the human or mammalian cells are chosen from one or more of human, porcine, or bovine cells.
